(19) United States
(12) Patent Application Publication
(10) Pub. No.: US 2009/0088757 A1
(43) Pub. Date: Apr. 2, 2009

(54) Acetabular reamer

(75) Inventor: Peter Tulkis, Paramus, NJ (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Howmedica Osteonics Corp., Mahwah, NJ (US)

(21) Appl. No.: 11/906,378

(22) Filed: Oct. 2, 2007

(51) Int. Cl. A61B 17/56

(52) U.S. Cl. 606/81

(57) ABSTRACT

A reamer assembly includes a tool driver and a tool. Tool driver comprises a shaft. The tool receiving end of the shaft has a flange. Formed in the flange are four L-shaped bayonet catches which receive the tool. The tool has a body having slots. A cutter can slidably fit in the slots. When installed in the slots, the cutters project slightly above the body. The projecting portions of the cutters form the cutting edges for shaving or cutting tissue. The reamer is formed by attaching a tool to the tool driver by lowering it in the L-shaped bayonet catches. The reamer can be coupled to a drive means such as a hand drill and inserted in a body cavity. Operating the reamer against body tissue results in shaving of body tissue.

### BACKGROUND OF THE INVENTION

In hip replacement surgery, to ensure a proper fit of the orthopedic device, deteriorated or diseased bone needs to be shaved away to healthy bone tissue so that the reamed acetabulum matches the contours of the implant to be fitted. An acetabular reamer is used for shaving the acetabulum, in preparation for implanting acetabular cup or socket. A rotary tool provides the motive force for the reamer. The rotary tool is attached to the base of the reamer via appropriate structure. The reamer base can include various backing plates, crossbars and pins which attach to the attachment structure connected with the rotary tool.

In a typical hip replacement procedure, a surgeon makes an incision in the hip area, displaces the existing hip joint, shapes the acetabulum with the reamer to receive a socket and inserts the socket. Removable reamer heads of increasingly larger size are used to enlarge the acetabulum during the reaming procedure. However, each time a larger reaming head is needed, the reamer must be removed from the patient, a larger reamer head is installed, and reinserted into position for further reaming. This procedure may be repeated several times until the acetabulum is completely prepared to fit the socket. However, the need for continued removal, change, and reinsertion of the reamer increases the time required to perform the operation, resulting in increased infection exposure. Thus there is a need for a reamer that is easy to attach to and to remove from the rotary tool that is used to drive the reamer.

### SUMMARY OF THE INVENTION

The present invention discloses a reamer assembly including a tool driver and a tool. Tool driver comprises a shaft. One end of the shaft is adapted to be connected to a drive means such as a rotary tool for example a hand drill or an electric motor. The other end of the shaft is adapted to receive the tool. The tool receiving end of the shaft has a flange. Formed in the flange are four L-shaped bayonet catches which receive the tool.

The tool has a body having slots. A cutter can slidably fit in the slots. The cutter may be selected from one of the several embodiments described herein. When installed in the slots, the cutters project slightly above the body. The projecting portions of the cutters form the cutting edges for shaving or cutting tissue. The body has openings formed next to slots to facilitate removal of tissue that is shaved by the cutters.

The cutter of first embodiment has a first blade and a second blade. The first blade has a semi-circular edge and a substantially straight edge that is divided in two sections by a slot. The slot extends from straight edge towards the semi-circular edge. Second blade also has a semi-circular edge and a substantially straight edge. A slot extends from semi-circular edge towards straight edge. The first blade and the second blade are assembled together to form the cutter. The assembly is formed by sliding the blades into each other such that the body of first blade is located in the slot in the second blade, and the body of second blade is located in the slot of the first blade. In this assembled state semi-circular edges of both the first and the second blades are located on a hemispherical surface.

The second, third and fourth embodiment of the cutter are substantially similar to the first embodiment and may be used in place of cutter of first embodiment. The significant difference between various embodiments of the cutter being in the portion that is received in L-shaped bayonet catches and the manner in which these portions are attached to the body of the blades. The cutter of the second, third or fourth embodiment are inserted in the slots in the body of the tool to form the tool. The tool formed using the cutter of the second, third or fourth embodiment are substantially similar to the tool formed using the cutter of the first embodiment except in the region that attaches to the tool driver.

The tool formed using the first, second, third or the fourth embodiment of the cutter is attached to the tool driver by lowering it in the L-shaped bayonet catches to form a reamer assembly. To attach the tool formed by using the cutter of the first, second or third embodiment, two sections (alternatively called “feet”) formed on one blade are captured in L-shaped bayonet catches thereby forming the reamer assembly. To attach the tool formed by using the cutter of the fourth embodiment, two sections formed on one blade and two feet formed on the other blade are captured in L-shaped bayonet catches thereby forming the reamer assembly. The reamer assembly can be coupled to a drive means such as a hand drill and inserted in a body cavity. Operating the reamer assembly against body tissue results in shaving of body tissue.

### BRIEF DESCRIPTION OF THE DRAWINGS

FIG. 1 is a perspective view of a reamer assembly.

FIG. 2 is a side view of a tool driver used in the reamer assembly of FIG. 1.

FIG. 3 is a perspective view of the bottom of tool body 40.

FIG. 3A is a perspective view of the bottom of tool body 40 with cuter of FIG. 7B inserted therein.

FIG. 4 is a perspective view of blades used to make cutter of FIG. 5.

FIG. 5 is a perspective view of the cutter made using blades of FIG. 4.

FIG. 6A is a perspective view of blades used to make cutter of FIG. 6B.

FIG. 6B is a perspective view of the cutter made using blades of FIG. 6A.

FIG. 7A is a perspective view of blades used to make cutter of FIG. 7B.

FIG. 7B is a perspective view of the cutter made using blades of FIG. 7A.

FIG. 8A is a perspective view of blades used to make cutter of FIG. 8B.

FIG. 8B is a perspective view of the cutter made using blades of FIG. 8A.

### DETAILED DESCRIPTION

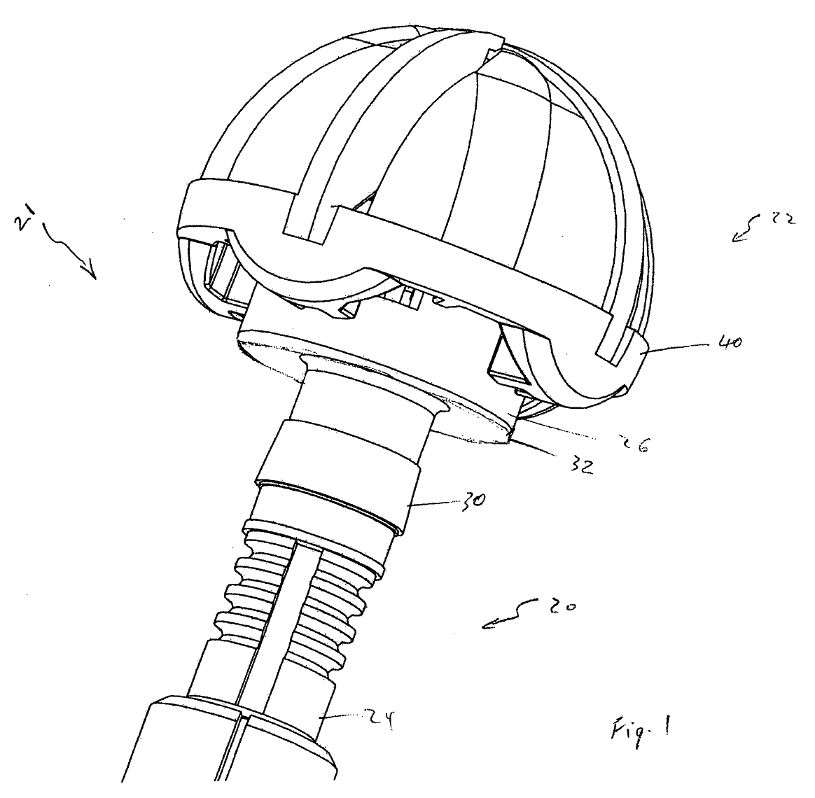

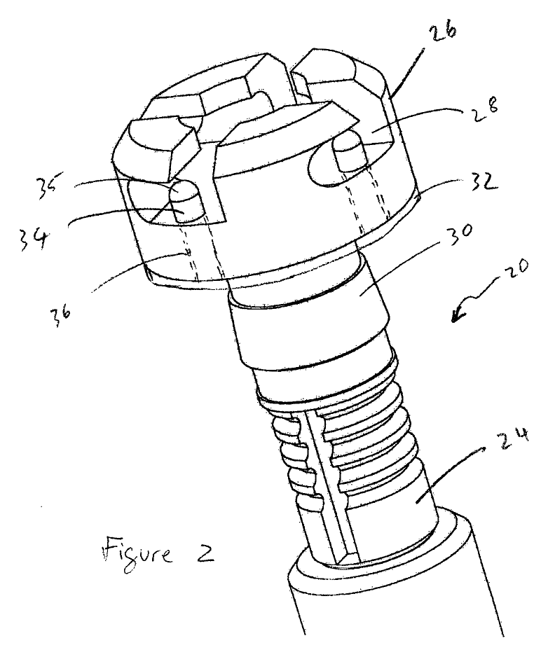

FIG. 1 shows a reamer assembly 21 including a tool driver 20 and a tool 22. FIG. 2 is an isometric view of tool driver 20. Tool driver 20 comprises a shaft 24. One end of shaft 24 is adapted to be connected to a drive means such as a hand drill or an electric motor. The other end of shaft 24 is adapted to receive tool 22. The tool receiving end of shaft 24 has a flange 26. Flange 26 forms a discontinuous cylindrical wall. Formed in flange 26 are four L-shaped bayonet catches 28 which are intended to receive tool 22 as discussed in detail hereafter.

A sleeve 30 is slidably mounted on shaft 24. Sleeve 30 has a flange 32 (FIG. 1) formed at one end. A spring (not shown) is located between sleeve 30 and shaft 24 such that flange 32 is urged towards flange 26. Four pins 34 project from surface of flange 32. Pins 34 are parallel to the shaft 24. Flange 26 has four holes 36. Pins 34 pass through these holes 36 in order to close bayonet catches 28 and thus lock tool 22 on tool driver 20. To open the bayonet catches, it suffices to distance sleeve 30 from flange 26 by pushing on sleeve 30. Pins 34 have a tapered area 35 formed in the region extending above surface of flange 26.

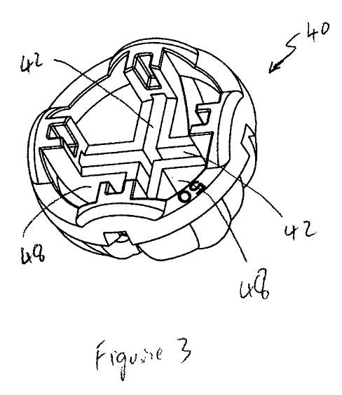

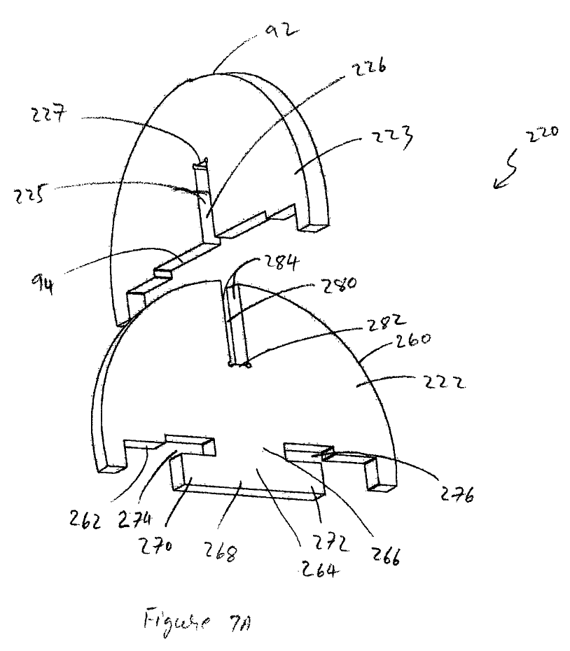

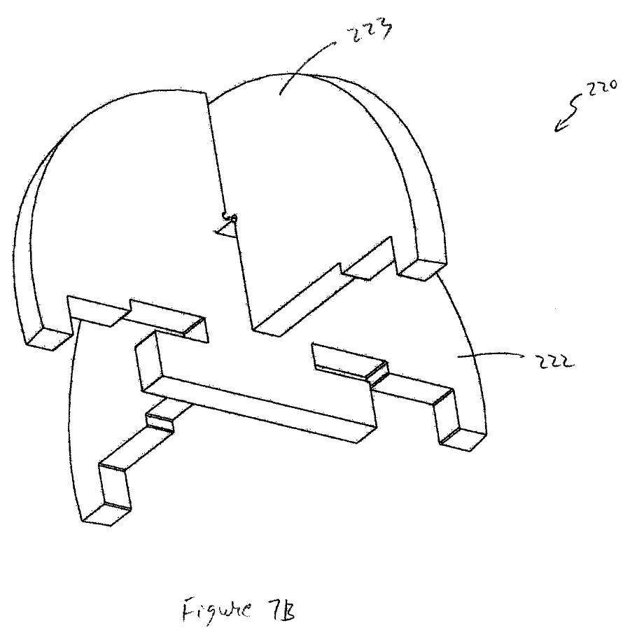

FIG. 3 is an isometric view of the bottom of tool body 40 of tool 22. FIG. 3A is an isometric view of the bottom of tool body 40 with cutter 220 (FIG. 7B) inserted therein. Body 40 may have hemispherical shape. Body 40 can have any other suitable shape. Body 40 is made from suitable material such as Polycarbonate or polypropylene or polyethylene or any other suitable material. Body 40 has slots 42. A cutter 220 (FIG. 3A) slidably fits in slots 42. Any other cutter described herein can similarly be inserted in slots 42. When installed in slots 42, cutter 220 project slightly above body 40, the projecting portions of cutter 220 form the cutting edges for shaving or cutting tissue. Body 40 has openings formed next to slots 42. These openings may facilitate removal of tissue that is shaved by cutter 44. The shaved tissue may be temporarily stored in chambers 48 formed in body 40. Chambers 48 may be emptied and cleaned after tool 22 is withdrawn from the body and tool 22 is separated from shaft 24.

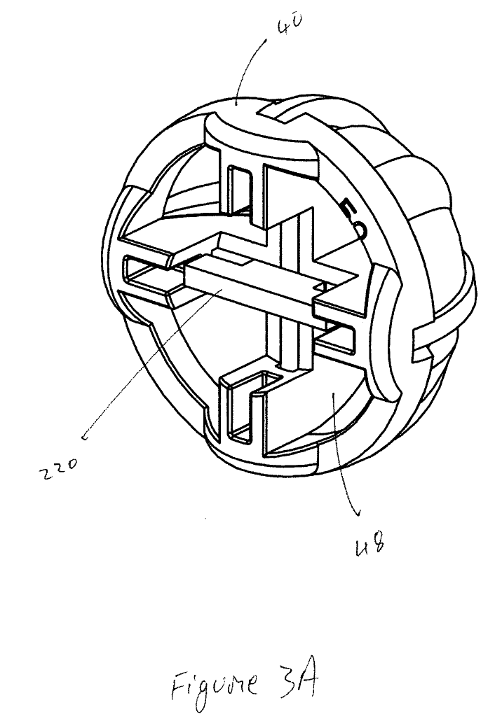

FIG. 4 shows a first blade 50 and a second blade 52 used to form cutter 44. First blade 50 has a semi-circular edge 54 and a substantially straight edge 56. Straight edge 56 extends in a direction away from the center of first blade 50. Straight edge 56 is divided in two horizontal sections 58 and 60 by a centrally located slot 62. Slot 62 extends from edge 56 towards the semi-circular edge 54. Slot 62 has a base 64 and side walls 66. Tabs 67 and 68 are formed at opposing ends of semi-circular edge 54. Slots 72 and 74 separate tabs 67 and 68 from sections 58 and 60 respectively. Sections 58 and 60 may have rectangular cross-sections sized to fit snugly in the L-shaped bayonet catches 28. Slot 72 has a first section 76 that is connected to a second section 78 by a narrowed opening 80. Similarly, slot 74 has a first section 82 that is connected to a second section 84 by a narrowed opening 86. Slots 72 and 74 allow sections 58 and 60 to be received in L-shaped bayonet catches 28. Second blade 52 has a semi-circular edge 92 and a substantially straight edge 94. Slot 96 extends from semi-circular edge 92 towards straight edge 94. Slot 96 has a base 98 and side walls 100. Tabs 106 and 108 are formed at opposing ends of semi-circular edge 92. Tabs 106 and 108 extend away from straight edge 94.

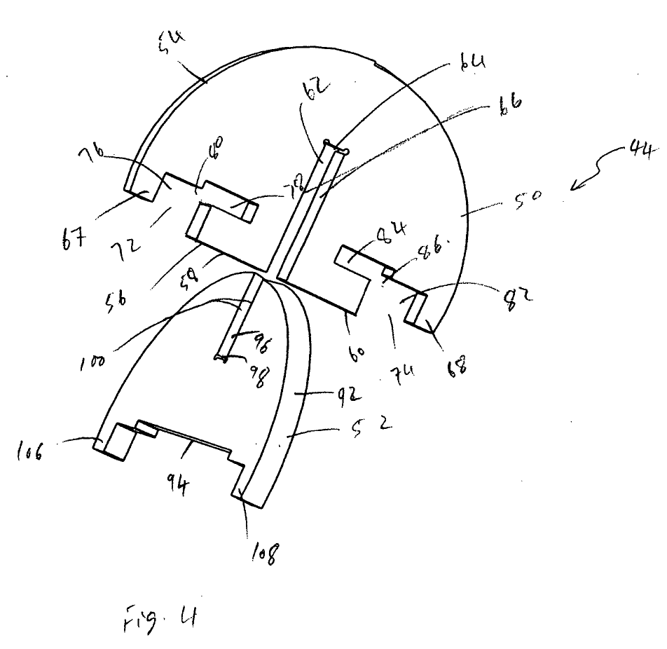

First blade 50 and second blade 52 are assembled together to form cutter 44 (FIG. 5). The assembly is formed by sliding the blades into each other such that the body of first blade 50 is located in slot 96, the body of second blade 52 is located in slot 62 and base 64 is adjacent base 98. In this assembled state semi-circular edge 54 and semi-circular edge 92 are located on a substantially hemispherical surface. Cutter 44 may be inserted in slots 42 in body 40.

To attach tool 22 to tool driver 20, tool 22 is lowered in L-shaped bayonet catches 28. Next, sleeve 28 is pushed such that pins 34 are retracted in flange 26 and tool 22 rotated such that sections 58 and 60 pass over pins 34. Next, sleeve 28 is released, which in turn results in pins 34 pushing through holes 36 and capturing sections 58 and 60 in L-shaped bayonet catches 28 thereby forming reamer assembly 21. Any two diametrically opposing L-shaped bayonet catches 28 are used to attach tool 22 to driver 20.

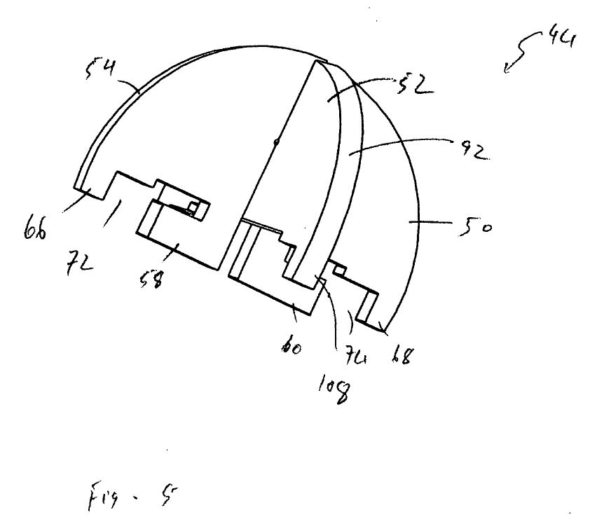

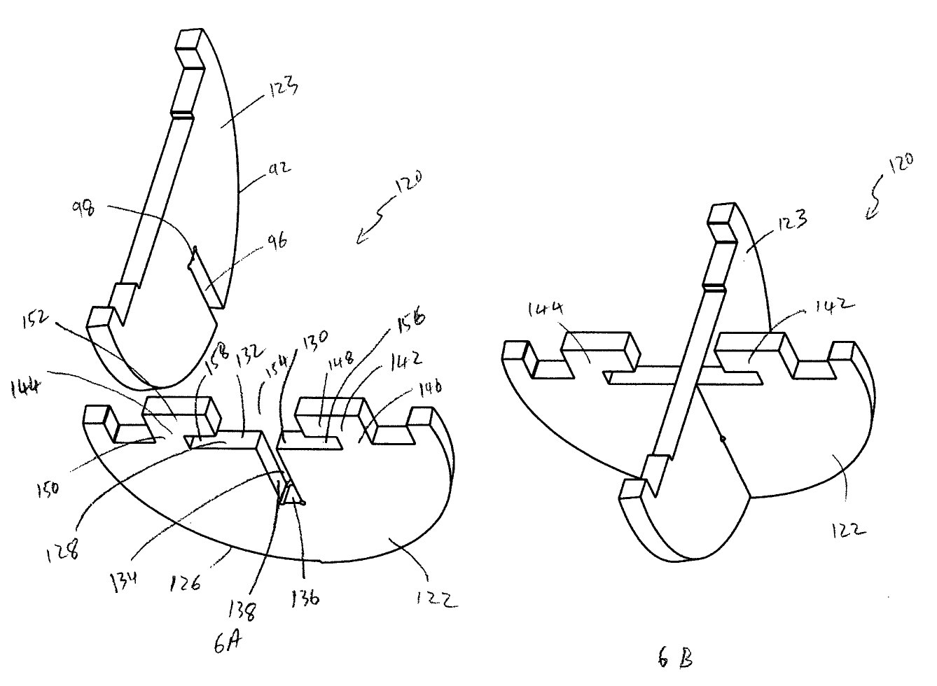

FIGS. 6A and 6B show a cutter 120. Cutter 120 may be use in place of cutter 44 described previously. FIG. 6A shows a first blade 122 and a second blade 123 in an unassembled state. FIG. 6B shows second blade 123 and first blade 122 assembled together forming cutter 120. Blade 123 is similar to blade 52 in construction. Features of blade 123 and blade 52 are designated by same numeral. Blade 122 has a semi-circular edge 126 and a substantially straight edge 128. Semi-circular edge 126 and straight edge 128 form a substantially “D” shaped body. Straight edge 128 is divided in two sections 130 and 132 by a centrally located slot 134. Slot 134 extends from straight edge 128 towards semi-circular edge 126. Slot 134 has a base 136 and side walls 138. Two “L” shaped feet 142 and 144 extend from sections 130 and 132 respectively. Feet 142 and 144 divide each of sections 130 and 132 respectively in two sub-sections. “L” shaped section 142 has a vertical portion 146 and a horizontal portion 148. “L” shaped section 144 also has a vertical portion 150 and a horizontal portion 152. A gap 154 is formed between horizontal portions 148 and 152. A gap 156 is also formed between horizontal portion 148 and straight edge 128 and a gap 158 is formed between horizontal portion 152 and straight edge 128.

First blade 122 and second blade 123 are assembled together to form cutter 120 (FIG. 6B). The assembly is formed by sliding the blades into each other such that the body of first blade 122 is located in slot 96, the body of second blade 123 is located in slot 134 and base 136 is adjacent base 98. In this assembled state semi-circular edge 126 and semi-circular edge 92 are located on a substantially hemispherical surface. Cutter 120 may be inserted in slots 42 in body 40 to form a second embodiment of tool 22.

To attach the second embodiment of tool 22 to tool driver 20, tool 22 is lowered in L-shaped bayonet catches 28. Next, sleeve 28 is pushed such that pins 34 are retracted in flange 26 and tool 22 rotated such that feet 142 and 144 pass over pins 34. Next, sleeve 28 is released, which in turn results in pins 34 pushing through holes 36 and capturing feet 142 and 144 in L-shaped bayonet catches 28 thereby forming reamer assembly 21.

FIGS. 7A and 7B show a cutter 220. Cutter 220 may be use in place of cutter 44 described previously. FIG. 7A shows a first blade 222 and a second blade 223 in an unassembled state. FIG. 7B shows second blade 223 and first blade 222 assembled together forming cutter 220. Blade 223 is similar to blade 52 in construction except for the location of slot 226. Slot 226 of second blade 223 replaces slot 96 of blade 52. Slot 226 extends from straight edge 94 towards the center of blade 223. Slot 226 has walls 225 and a base 227. First blade 222 has a semi-circular edge 260 and a stepped edge 262. Semi-circular edge 260 and stepped edge 262 form a substantially “D” shaped body. An inverted “T” shaped foot 264 extends from stepped edge 262. Foot 264 has a vertical portion 266 and a horizontal portion 268. Horizontal portion 268 has a first section 270 and a second section 272. A gap 274 is formed between section 270 and straight edge 262 and a gap 276 is formed between section 272 and straight edge 262. First blade 222 has a Slot 280 that extends from semi-circular edge 260 towards straight edge 262. Slot 280 has a base 282 and side walls 284.

First blade 222 and second blade 223 are assembled together to form cutter 220 (FIG. 7B). The assembly is formed by sliding the blades into each other such that the body of first blade 222 is located in slot 226, the body of second blade 223 is located in slot 280 and base 282 is adjacent base 227. In this assembled state semi-circular edge 260 and semi-circular edge 92 are located on a substantially hemispherical surface. Cutter 220 may be inserted in slots 42 in body 40 to form a third embodiment of tool 22.

To attach the third embodiment of tool 22 to tool driver 20, tool 22 is lowered in L-shaped bayonet catches 28. Next, sleeve 30 is pushed such that pins 34 are retracted in flange 26 and tool 22 rotated such that sections 270 and 272 pass over pins 34. Next, sleeve 30 is released, which in turn results in pins 34 pushing through holes 36 and capturing sections 270 and 272 in L-shaped bayonet catches 28 thereby forming reamer assembly 21.

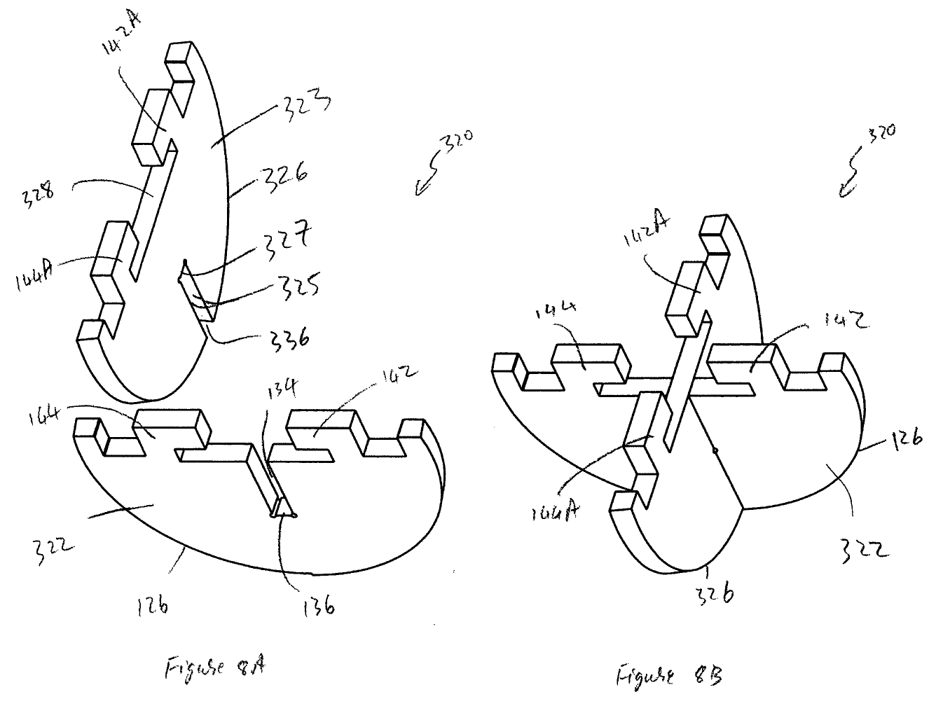

FIGS. 8A and 8B show a cutter 320. Cutter 320 may be use in place of cutter 44 described previously. FIG. 8A shows a first blade 322 and a second blade 323 in an unassembled state. FIG. 8B shows second blade 323 and first blade 322 assembled together forming cutter 320. Second blade 323 has a semi-circular edge 326 and a substantially straight edge 328. Semi-circular edge 326 and straight edge 328 form a substantially “D” shaped body. Two “L” shaped sections 142A and 144A extend from straight edge 328. A slot 336 extends from semicircular edge 326 towards the center of blade 323. Slot 326 has walls 325 and a base 327. Construction of first blade 322 is similar to previously described blade 122. Features common to both blade 122 and blade 322 are designated by same numerals in FIGS. 6 and 8 respectively.

First blade 322 and second blade 323 are assembled together to form cutter 320 (FIG. 8B). The assembly is formed by sliding the blades into each other such that the body of first blade 322 is located in slot 336, the body of second blade 323 is located in slot 134 and base 327 is adjacent base 136. In this assembled state semi-circular edge 326 and semi-circular edge 126 are located on a substantially hemispherical surface. Cutter 320 may be inserted in slots 42 in body 40 to form a fourth embodiment of tool 22.

To attach the fourth embodiment of tool 22 to tool driver 20, tool 22 is lowered in L-shaped bayonet catches 28. Next, sleeve 30 is pushed such that pins 34 are retracted in flange 26 and tool 22 rotated such that sections 142, 142A, 144 and 144A pass over pins 34. Next, sleeve 30 is released, which in turn results in pins 34 pushing through holes 36 and capturing feet 142, 142A, 144 and 144A in L-shaped bayonet catches 28 thereby forming reamer assembly 21.

Reamer assembly 21 can be coupled to a drive means such as a hand drill or any other means used in the operating room that are known to one skilled in art. Reamer assembly 21 is inserted in a body cavity of the patient in any manner known to one skilled in art and operated by powering the drive means. Operating reamer assembly 21 against body tissue results in shaving of body tissue that may enter chambers 48 via openings 46. Upon withdrawal of reamer assembly from the body cavity, tool 22 may be dismantled from tool driver 20 and shaved tissue may be removed from chambers 48.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A tissue reamer comprising:
a first blade, the first blade having a first semi-circular edge and a first slot extending from the straight edge towards the first semi-circular edge;
a second blade, the second blade having a second semi-circular edge and a second slot extending from the second semi-circular edge towards the center of the second blade, the first blade and the second blade being assembled together by sliding the first slot in the second slot; and
a substantially hemispherical body having openings formed therein such that when the assembly of the first and the second blade is inserted in the openings the first semi-circular edge and the second semi-circular edge projects above the surface of the hemispherical body to form the cutting edge for cutting the tissue.

2. The tissue reamer of claim 1, further comprising:
a first tab and a second tab formed at the opposing end of the first semi-circular edge of the first blade;
a third tab and a fourth tab formed at the opposing end of the second semi-circular edge of the second blade; and
a circular member formed at the end of the hemispherical body having the largest diameter, the circular member having at least one notch capable of receiving at least one of the first, second, third and fourth tab.

3. The tissue reamer of claim 1, further comprising:
a first straight edge formed on the first blade; and
a first and a second section attached to and spaced from the first straight edge.

4. The tissue reamer of claim 3, wherein the first section and the second section extend away from each other.

5. The tissue reamer of claim 3, wherein the first section and the second section extend towards each other.

6. The tissue reamer of claim 3, further comprising:
a shaft having fourth end, the fourth end of the shaft being adapted to lock with the first section and the second section thereby attaching the first and the second blades to the shaft.

7. The tissue reamer of claim 3, further comprising:
a second straight edge formed on the second blade; and
a first foot and a second foot attached to and spaced from the second straight edge of the second blade.

8. The tissue reamer of claim 7, wherein at least one of the first and second foot and the first and second section have a rectangular cross-section.

9. The tissue reamer of claim 7, wherein the first foot and the second foot extend towards each other.

10. The tissue reamer of claim 7, wherein the first foot and the second foot extend away from each other.

11. The tissue reamer of claim 7, further comprising: a shaft having fourth end, the fourth end of the shaft being adapted to lock with the first section, the second section, the first foot and the second foot thereby attaching the reamer head to the shaft.

12. The tissue reamer of claim 6, wherein the shaft has a fifth end adapted to couple with a rotary tool.

13. The tissue reamer of claim 12, wherein the shaft further comprises spring loaded bayonet latches that operate to lock the first section, the second section, the first foot and the second foot thereby attaching the first and the second blades to the shaft.

14. A reamer for reaming a bone cavity, comprising;
a first blade, the first blade having a first semi-circular edge, a first horizontal section, a second horizontal section and a first tab formed at each end of the first semicircular edge and a first slot extending between the first straight edge and the second straight edge and towards the first semi-circular edge;
a second blade, the second blade having a second semi-circular edge and a second tab formed at each end of the second semi-circular edge and a second slot extending from the second semi-circular edge towards the center of the second blade, the first blade and the second blade being assembled together by sliding the first slot in the second slot; and
a substantially hemispherical body having openings formed therein such that when the assembly of the first and the second blade is inserted in the openings the first semi-circular edges and the second semi-circular edge projects above the surface of the hemispherical body to form the reamer head.

15. The reamer of claim 14, further comprising:
a first foot attached to the second blade, the first foot having a third horizontal section; and
a second foot attached to the second blade, the second foot having a fourth horizontal section.

16. The reamer of claim 15, wherein at least one of the first, second, third and fourth horizontal section has a rectangular cross-section.

17. The reamer of claim 15, further comprising:
a circular member formed at the end of the hemispherical body having the largest diameter, the circular member having notches capable of receiving the first tabs and the second tabs.

18. The reamer of claim 14, further comprising:
a shaft having fourth end, the fourth end of the shaft being adapted to lock with the first horizontal section and the second horizontal section thereby attaching the reamer head to the shaft.

19. The reamer of claim 15, further comprising:
a shaft having fourth end, the fourth end of the shaft being adapted to lock with the first horizontal section, the second horizontal section, the third horizontal section and the fourth horizontal section thereby attaching the reamer head to the shaft.

20. The reamer of claim 18, wherein the shaft has a fifth end adapted to couple with a rotary tool.

21. The reamer of claim 19, wherein the shaft further comprises spring loaded bayonet latches that operate to lock the first horizontal section, the second horizontal section, the third horizontal section and the fourth horizontal section thereby attaching the first and the second blades and the reamer to the shaft.
